(19) United States
(12) Patent Application Publication
(10) Pub. No.: US 2025/0107830 A1
(43) Pub. Date: Apr. 3, 2025

(54) Indicating, Auto-Resetting Head Inserter

(71) Applicant: K2M, Inc., Leesburg, VA (US)

(72) Inventors: Eric Cecco, Purcellville, VA (US); Nicholas Padovani, Fairfax, VA (US); Tyler Clarkson, McLean, VA (US); Michael Barrus, Redondo Beach, CA (US); Steven Greendyk, Butler, NJ (US)

(21) Appl. No.: 18/895,716

(22) Filed: Sep. 25, 2024

Related U.S. Application Data
(60) Provisional application No. 63/541,975, filed on Oct. 2, 2023.

(51) Int. Cl. A61B 17/70

(52) U.S. Cl. CPC A61B 17/7082

(57) ABSTRACT

A surgical instrument for inserting and attaching a modular tulip head to a bone screw that includes an outer sleeve, an indicator button, a shaft, and an auto-resetting component. The outer sleeve has a distal end and a proximal end, and the distal end is configured to receive a modular tulip head. The indicator button is adjacent to the proximal end of the outer sleeve and movable between a first position and a second position. The shaft is slidably disposed within the outer sleeve, and the auto-resetting component is slidably linked to the indicator button and configured to automatically reposition the indicator button to the first position.

### CROSS-REFERENCE TO RELATED APPLICATIONS

This application claims the benefit of the filing date of U.S. Provisional Patent Application No. 63/541,975 filed on Oct. 2, 2023, the disclosure of which is hereby incorporated by reference in its entirety.

### BACKGROUND

Bone fixation hardware plays a critical role in promoting the healing and fusion of bone in surgical operations. For instance, polyaxial pedicle screws and rods are commonly utilized in spinal fusion procedures. Because proper implantation of such hardware is critical to obtaining desired results, medical personnel use surgical tools specifically designed for this purpose. For example, an inserter is typically used to insert and implant pedicle screws.

One type of pedicle screw that presents additional issues in insertion and implantation are modular pedicle screws, which require a modular tulip head to be attached to an already implanted screw portion. Attempts have been made to develop inserters that provide feedback indicating the tulip head has been properly attached to the screw. However, such inserters have shown to be susceptible to false positive feedback of proper insertion along with users failing to reset the indicator mechanisms.

As such, further improvements are desired in the field of surgical tools for inserting and attaching modular tulip heads to bone screws. Particularly, there is a need for an inserter that provides reliable means to ensure proper connection between modular tulip heads and implanted screw portions and assists in resetting indicator components.

### BRIEF SUMMARY

Some aspects herein relate to a surgical instrument for placing and attaching a modular tulip head onto an implanted bone screw or implant with an attachment indicator to confirm the attachment of the tulip head and an automatic reset mechanism for the attachment indicator. The automatic reset mechanism having a ramp that slidably engages the attachment indicator when the instrument releases the tulip head such that the attachment indicator is repositioned to a non-indicating position. After being repositioned to the non-indicator position, the attachment indicator is further engaged by a second component to hold the attachment indicator in the non-indicator position until triggered by a feedback mechanism. The attachment indicator may also be manually reset and re-triggered without unlocking the tulip head from the instrument to reconfirm proper attachment of the tulip head.

In another aspect, the present disclosure relates to a surgical instrument for inserting and attaching a modular tulip head to a bone screw or implant assembly. The surgical instrument may include an outer sleeve, an indicator button, a shaft slidably disposed within the outer sleeve, and an auto-resetting component slidably linked to the indicator button and the shaft. The auto-resetting component may be configured to automatically reposition the indicator button to the first position. The outer sleeve may have a proximal end and a distal end configured to receive the tulip head. The indicator button may be adjacent the proximal and movable between a first position and a second position. The shaft may be slidably disposed within the outer sleeve. The auto-resetting component may include a reset ramp. The reset ramp may be slidably disposed within an aperture of the indicator button. The auto-resetting component may be disposed within the outer sleeve. Alternatively, the auto-resetting component may be disposed external to the outer sleeve of the surgical instrument. The surgical instrument may further include an inner sleeve disposed between the outer sleeve and the shaft such that the shaft is slidably disposed within the inner sleeve. The auto-resetting component may include connector arms extending distally from the auto-resetting component such that the connector arms attach to a proximal side of the inner sleeve. The indicator button may include an internal portion and an indicating portion. The internal portion may define the aperture of the indicator button. The indicator button may be attached to an indicator spring. The indicator button and the indicator spring may be positioned orthogonal to a longitudinal axis of the outer sleeve. 11.

The surgical instrument may further include a slot pin disposed within an elongated slot of the outer sleeve. The elongated slot configured to receive the slot pin such that the slot pin extends therethrough and engages the inner sleeve. The inner sleeve may have a pin hole configured to receive the slot pin. The surgical instrument may further include a knob slidably disposed around a portion of the outer sleeve above the slot such that the knob is connected to the inner sleeve via the slot pin. The knob may be configured to slide the inner sleeve along a longitudinal axis of the outer sleeve based on the length of the slot. The surgical instrument may further include a setting shaft disposed within a hollow chamber of a handle. The handle may be attached to the outer sleeve such that the setting shaft is partially disposed within the outer sleeve. The setting shaft may include a setting cap extending distally from the setting shaft into the outer sleeve. The setting cap may have a lip configured to be inserted into the aperture of the indicator button when the indicator button is in the non-indicating position. The proximal end of the shaft may be connected to the setting cap such that the shaft extends through the aperture of the indicator button. The surgical instrument may further include a holding spring disposed around the setting shaft adjacent to the setting cap. The holding spring may be configured to push the lip of the setting cap into the aperture of the indicator button when the indicator is in the non-indicating position. The outer sleeve may include a side hole that is in communication with the hollow annulus of the outer sleeve. The side hole being dimensioned such that the indicator button can be inserted therein. The inner sleeve may include at least two actuating arms extending from the distal end of the inner sleeve configured to engage an inner surface of the tulip head. The hollow annulus of the outer sleeve may include an inner shoulder. The inner shoulder of the hollow annulus may be disposed near the proximal opening such that the inner shoulder limits the movement of the ramp in the distal direction.

In another aspect, the present disclosure relates to a kit may include an inserter as described herein, a plurality of modular tulip heads, and a plurality of bone screws. In some instances, some of the plurality of bone screws may be double threaded.

In yet another aspect, the present disclosure relates to inserting a modular tulip head using an inserter by a process. In the process, tulip head may be engaged with the inserter. The tulip head may then be placed onto a bone screw or implant assembly. An indicator button may be triggered to move to an indicating position after placement of the tulip head on the bone screw or implant assembly. A knob of the inserter may then be retracted to an original position after the indicator button is triggered such that the indicator button is automatically reset to a non-indicating position by a reset component. The indicator button may be automatically reset by sliding the reset component into an aperture of the indicator button. Engaging the tulip head with the inserter may include the inner sleeve being slid in a distal direction along the longitudinal axis of the outer sleeve. Retracting the knob may include the knob being slid along the outer sleeve until the knob abuts a flange. The knob may be disposed around the outer sleeve and connected to the inner sleeve. Engaging the tulip head with the inserter may include the knob being pushed in a distal direction. Engaging of the tulip head with the inserter may be done by arms being actuated to engage an undercut or groove feature on the tulip head, the arms extending from the inner sleeve. Triggering of the indicator button may be done by a setting shaft being displaced. The setting shaft may be arranged to hold the indicator button in the non-indicating position while partially inserted into an aperture of the indicator button. Displacing of the setting shaft may be caused by an indicator shaft being shifted in the distal direction due to contact with the bone screw. Retracting of the inner sleeve may cause the reset component to engage the aperture of the indicator button.

The process may further include, after indicator button is reset to the original position, a distal lip being inserted into the aperture of the indicator button. The process may further include the tulip head being attached to a bone screw before triggering the indicator button. The indicator button may be triggered by pulling a handle of the inserter away from the tulip head.

In some instances, the process may relate to confirming attachment of a tulip head by additional process steps. The process steps may include the indicator button being moved to the non-indicating position. The handle being pushed toward the tulip head to restore the handle to previous position. The handle being pulled away from the tulip head to repeat triggering the indicator button.

In other aspects, the present disclosure relates to a surgical instrument for inserting and attaching a tulip head to a pedicle screw that may include a sleeve, and indicator button, an inner shaft, a knob, and a reset ramp. The sleeve may have a proximal end and a distal end configured to receive the tulip head. The indicator button may be configured to actuate between an indicating position and a non-indicating position. The inner shaft may be slidably disposed within the sleeve and configured to engage the tulip head. The knob may be slidably disposed around the sleeve. The reset ramp may be linked to the knob and configured to automatically reposition the indicator button from the indicating position to the non-indicating position. The reset ramp may be disposed external to the sleeve. The surgical instrument may further include a handle slidably connected to the sleeve. The indicator button may partially disposed in a side hole of the handle. The handle may include an internal spring that resists proximal movement of the handle with respect to the sleeve. The knob may be connected to the inner shaft via a slot pin that extends through an elongated slot defined by the sleeve. The short region of the reset ramp may be aligned with an aperture of the indicator button. The aperture of the indicator button may be configured to receive the reset ramp in both the indicating position and the non-indicating position. The reset ramp may include a first ramp and a second ramp. The first and second ramps may be disposed on opposite sides of the sleeve. The first and second ramps may define the same shape. The sleeve may include a distal sleeve and a proximal second sleeve. The narrow region may be disposed between the proximal and distal sleeves. The inner shaft may include at least two arms extending from a distal end of the inner shaft. The arms may be configured to expand laterally outward to engage a modular tulip head. When the indicator button is in the non-indicating position, the aperture of the indicator button may be centered around a longitudinal axis of the tubular cavity of the handle. The surgical instrument may further include an actuating spring that may enclosed within the proximal sleeve. The actuating spring may be indirectly linked to a handle. The surgical instrument further include a second reset ramp connect to the rest ramp via an annular ring.

### BRIEF DESCRIPTION OF THE DRAWINGS

A more complete appreciation of the subject matter of the present disclosure and the various advantages thereof may be realized by reference to the following detailed description which refers to the accompanying drawings, in which:

FIG. 1 is a perspective view of an inserter in accordance with an embodiment of the present disclosure;

FIG. 2 is a side view of the inserter of FIG. 1;

FIG. 3 is an exploded view of the inserter of FIG. 1;

FIG. 4 is a side view of the indicator button of the inserter of FIG. 1;

FIG. 5 is a side view of the reset ramp of the inserter of FIG. 1;

FIG. 6 is a side view of an outer sleeve of the inserter of FIG. 1;

FIG. 7 is a cross-sectional view of the outer sleeve of FIG. 12 across line D-D;

FIG. 8 is a side view of a knob of the inserter of FIG. 1;

FIG. 9 is a cross-sectional view of the knob of FIG. 8 across line F-F;

FIG. 10 is a cross-sectional view of the inserter of FIG. 2 across line A-A with the indicator button in a non-indicating position and the knob in the locked position;

FIG. 11 is a cross-sectional view of the inserter of FIG. 2 across line A-A with the indicator button in a non-indicating position and the knob in the locked position;

FIG. 12 is a cross-sectional view of the inserter of FIG. 2 across line A-A with the indicator button in an indicating position and the knob in the locked position;

FIG. 13 is a cross-sectional view of the inserter of FIG. 2 across line A-A with the indicator button in a non-indicating position and the knob in the unlocked position;

FIG. 14A is a cross-sectional view of the inserter of FIG. 2 across line A-A with a modular tulip head attached to the end of the inserter;

FIG. 14B is a cross-sectional view of the inserter of FIG. 2 across line A-A with a bone screw inserted into the distal end of the inserter and indicator button in an indicating position;

FIG. 14C is a cross-sectional view of the inserter FIG. 2 across line A-A, the inserter being in the unlocked position and attachment arms inserted into a tulip head;

FIG. 14D is a close-up view of the distal end of the inserter of FIG. 14C;

FIG. 14E is a cross-sectional view of the inserter of FIG. 2 across line A-A, the inserter being in the locked position and attached to a tulip head;

FIG. 14F is a cross-sectional view of the distal end of the inserter of FIG. 14E;

FIG. 15A is an enlarged cross-sectional view of the inserter of FIG. 2 across line A-A with the indicator button in a non-indicating position and the knob in an unlocked position

FIG. 15B is an enlarged cross-sectional view of the inserter of FIG. 2 across line A-A with the indicator button transitioning between an indicating position and a non-indicating position;

FIG. 16 is an enlarged cross-sectional view of the inserter of FIG. 2 across line A-A with the indicator button in an indicating position and the knob in a locked position;

FIG. 17 is an enlarged cross-sectional view of the inserter of FIG. 2 across line A-A with the indicator button in a non-indicating position and the knob in a locked position;

FIG. 18 is a perspective view of an inserter in accordance with another embodiment of the present disclosure;

FIG. 19. is a side view of the inserter of FIG. 18;

FIG. 20 is an exploded view of the inserter of FIG. 18;

FIG. 21 is an exploded view of an outer sleeve of the inserter of FIG. 18;

FIG. 22 is a top view of a reset ramp of the inserter of FIG. 18;

FIG. 23 is a side view of the reset ramp of FIG. 22;

FIG. 24 is a side view of the of the inserter of FIG. 18 without a handle and with an indicator button in an indicating position;

FIG. 25 is a side view of the inserter FIG. 18 without the handle and with the indicator button between the indicating position and a non-indicating position;

FIG. 26 is a side view of the inserter FIG. 18 without a handle and with the indicator button positioned in the non-indicating position;

FIG. 27 is a cross-sectional view of the inserter of FIG. 19 across line B-B with an indicator button in a non-indicating position and a knob in an extended position;

FIG. 28 is a cross-sectional view of the handle of the inserter of FIG. 19 across line B-B with the indicator button in an indicating position;

FIG. 29 is a cross-sectional view of the handle of the inserter of FIG. 19 across line B-B with the indicator button in the non-indicating position and the knob in a retracted position;

FIG. 30 is a close-up view of the handle of the inserter of FIG. 24;

FIG. 31 is a close-up view of the handle of the inserter of FIG. 25; and

FIG. 32 is a close-up view of the handle of the inserter of FIG. 26.

### DETAILED DESCRIPTION

As used herein unless stated otherwise, the term “anterior” means toward the front part of the body, and the term “posterior” means toward the back part of the body. When referring to specific directions in the following discussion of a certain device, the terms “proximal” and “distal” are to be understood in regard to the device's orientation and position during exemplary application to human body. Thus, the term “proximal” means closer to the operator or in a direction toward the operator, and the term “distal” means more distant from the operator or in a direction away from the operator. In addition, the terms “about,” “generally,” and “substantially” are intended to mean that deviations from absolute are included within the scope of the term so modified.

In a first aspect, the present disclosure relates to an inserter, as shown in the figures, capable of attaching to a modular tulip head to a bone screw and to confirm that attachment through an attachment indicator. The inserter includes, among other things discussed in more detail below, an auto-resetting mechanism that automatically resets the indicator to a non-indicating position after the indicator has been triggered to an indicating position. In the non-indicating position, an indicator (e.g., indicator button 30, 130 shown in the figures) is completely disposed within the inserter such that a top surface of the indicator is flush with the exterior surface of the inserter. In the indicating position, the indicator is only partially disposed within the inserter such that the remainder of the indicator protrudes above and beyond the exterior surface of the inserter. By eliminating the need to manually reset the indicator to the non-indicating position, the auto-resetting mechanism allows surgeons to more effectively and efficiently insert and attach multiple modular tulip heads to implanted bone screws.

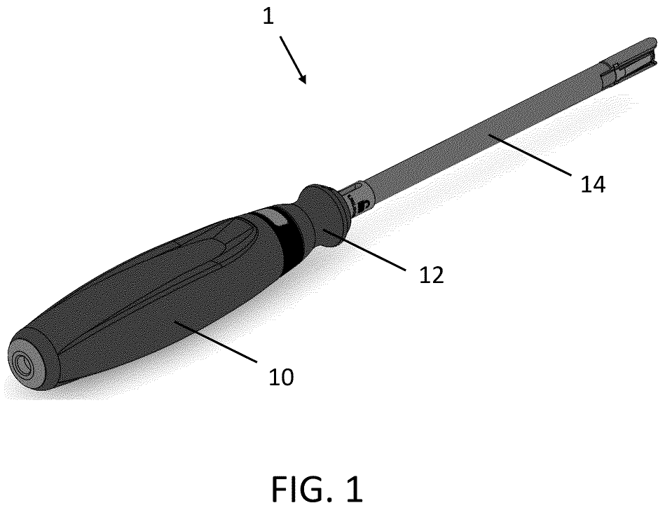

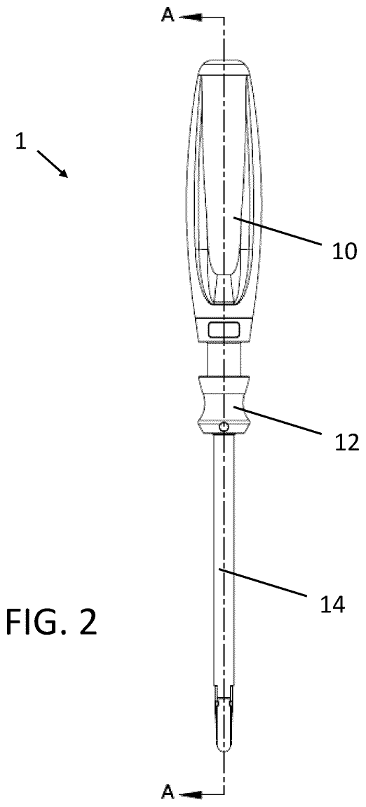

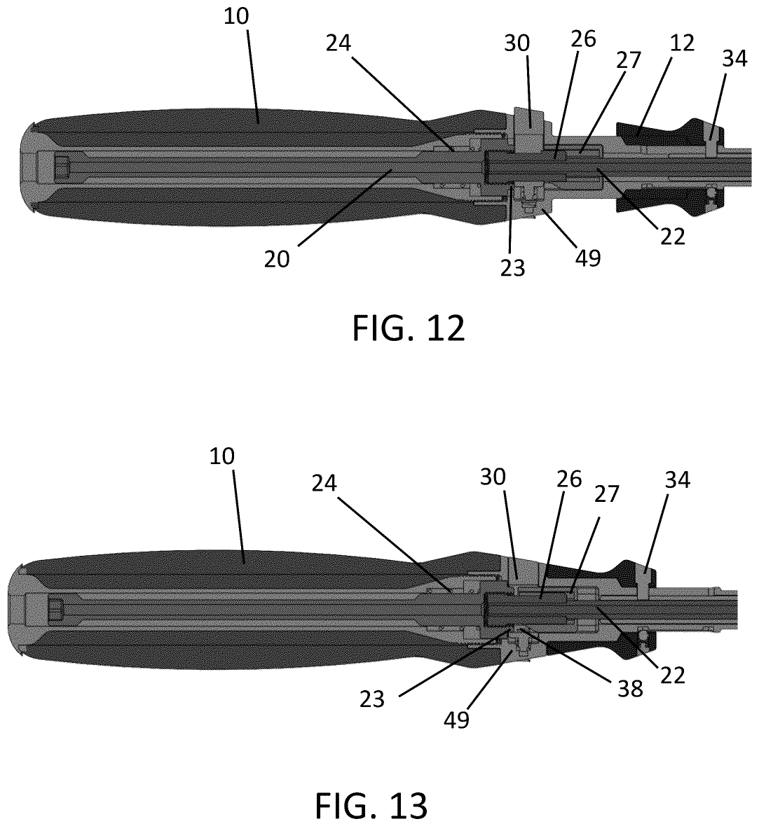

In one embodiment, as shown in FIGS. 1-17, an inserter 1 includes a handle 10 connected to an outer sleeve 14, an inner sleeve 18 slidably disposed within the outer sleeve and encasing a portion of an indicator shaft 22, a knob 12 slidably disposed around the outer sleeve, an indicator button 30 having an interior aperture 38 positioned proximal to the inner sleeve, and an auto-resetting component 27 attached to a distal end of the inner sleeve. Handle 10 is a tube that includes a hollow insert 32, a setting spring 24, and a setting shaft 20 disposed within the setting spring. Both setting spring 24 and setting shaft 20 are disposed within hollow insert 32 which is positioned within the tube formation of handle 10. Setting shaft 20 has a lip portion 23 extending from a setting cap 21 such that the lip portion is disposed within interior aperture 38 of indicator button 30 when the indicator button is in the non-indicating position. Hollow insert 32 has a first aperture 33 and a second aperture 31 located at the proximal and distal ends, respectively, such that the second aperture defines an inner diameter larger than that of the first aperture and the outer diameter of setting spring 24. Hollow insert 32 also includes an external threaded portion 37 on the exterior of a distal end of the hollow insert, the threaded portion corresponding to threading on outer sleeve 14 such that the outer sleeve and handle 10 can connect via the hollow insert, discussed in more detail below.

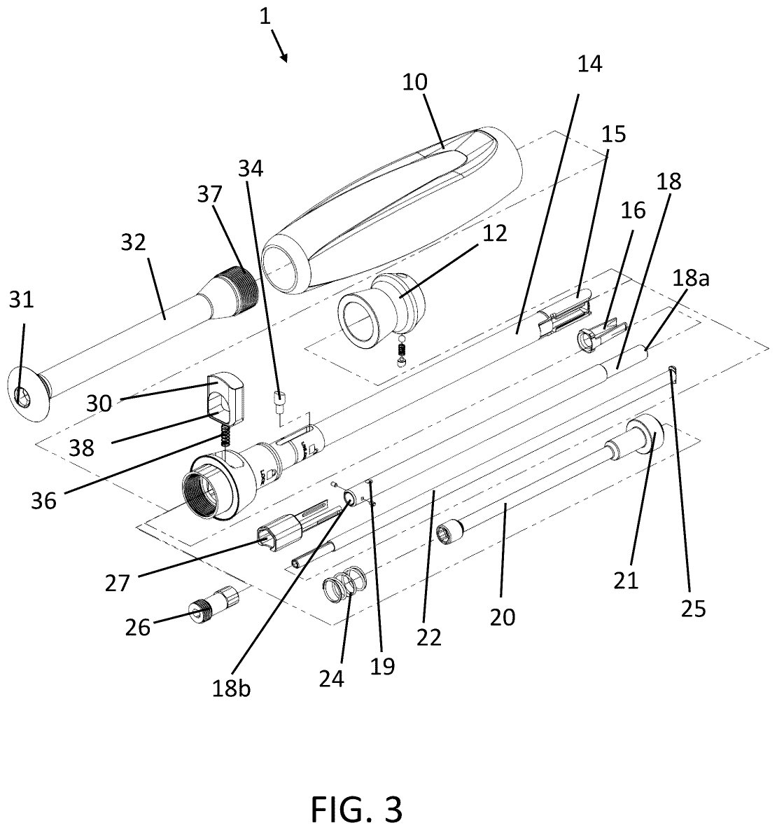

As best shown in FIG. 3, inner sleeve 18 is hollow and includes a distal opening 18a and a proximal opening 18b. Attachment arms 16 attach to and extend from the distal end of the inner sleeve. Attachment arms 16 are flexible such that the attachment arms can splay laterally outward. The distal end of attachment arms 16 are designed to engage with an interior region of a modular tulip head when the attachment arms are in an expanded state.

Indicator shaft 22 is a cylindrical rod that includes an alignment tube 26 and lateral arms 25 disposed at opposite ends. Lateral arms 25 are disposed at a distal end of indicator shaft 22 such that they are perpendicular to the longitudinal axis of the indicator shaft. A distal portion of indicator shaft 22 is disposed within inner shaft 18, and a proximal portion of the indicator shaft extends away from and is external to the inner sleeve due to the indicator shaft being longer than the inner sleeve. Alignment tube 26 is disposed at a proximal end of indicator shaft 22, and a threaded portion of alignment tube 26 is threadably attached to the distal end (i.e., setting cap 21) of setting shaft 20.

Auto-resetting component 27 is a sheath-like structure having one side that is diagonal to the others in a manner that forms a ramp feature 28 on the topside of the auto-resetting component. In other words, auto-resetting component 27 defines a ramp-like profile, as best shown in FIG. 5. The sheath-like structure of auto-resetting component 27 defines a through-hole such that alignment tube 26 can be positioned therein. Additionally, auto-resetting component 27 includes extension arms 29 that extend distally toward inner sleeve 18 and connect the auto-resetting component to the inner sleeve. Extension arms 29 include openings such that the extension arms can be pinned to the inner sleeve. Alternatively, however, auto-resetting component may be connected inner sleeve in a variety of ways, e.g., but not limited to, welding, threading, press fitting, clipping, and adhering thereon. In other embodiments, extension arms 29 may have extensions or protrusions that are received in holes in inner sleeve 18 to connect the extension arms to the inner sleeve.

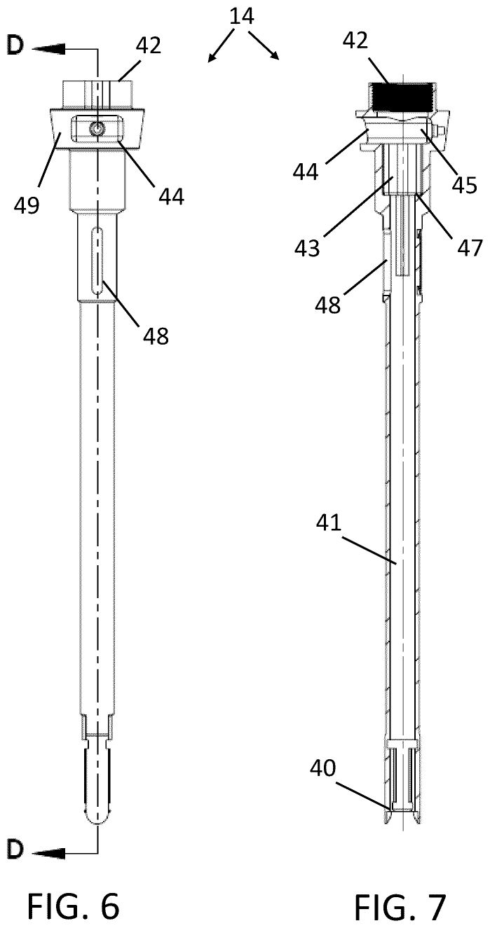

As shown in FIGS. 6-7, outer sleeve 14 includes a slotted opening 40 at the distal end of the outer sleeve, a threaded opening 42 at the proximal end of the outer sleeve, a side opening 44 adjacent to the threaded opening, and an elongated opening 48 disposed along the length of the outer sleeve near the side opening. Slotted opening 40, in other words, defines the opening of outer sleeve 14 that extends between distal arms 15 of the outer sleeve. Threaded opening 42 defines a larger diameter than slotted opening 40 such that externally threaded portion 37 of hollow insert 32 can be threadably attached to outer sleeve 14 and thereby connect handle 10 to the outer sleeve via the hollow insert. The interior of outer sleeve 14 defines an inner sleeve compartment 41, a ramp compartment 43 that has a larger inner diameter than the sleeve compartment, and an indicator compartment 45 that extends from side opening 44 into the outer sleeve perpendicular to the longitudinal axis of the outer sleeve. All the compartments of outer sleeve 14 are in communication with each other such that a shoulder 47 is formed between inner sleeve compartment 41 and ramp compartment 43. The exterior of outer sleeve 14 defines a flange 49 protruding radially outward from the outer sleeve around side opening 44.

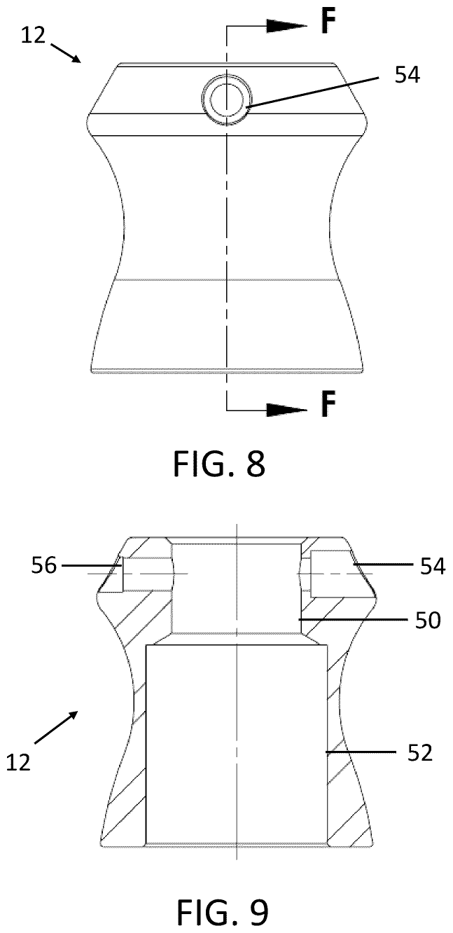

As shown in FIGS. 8-9, knob 12 is a tube that has a minor region 50, a major region 52, a large pin hole 54, and a small pin hole 56, the minor region having a smaller inner diameter than that of the major region. Both small pin hole 56 and large pin holes 54 extend through the sidewall of the tube structure of the knob and into the minor region. When knob 12 is placed in the retracted (i.e., unlocked) position next to flange 49, major region 52 is disposed around ramp compartment 43 of outer sleeve 14, and minor region 50 is disposed around inner sleeve compartment 41 of the outer sleeve such that large pin hole 54 is aligned with elongated opening 48 and a pin hole 19 of inner sleeve 18. A pin 34 is disposed within large pin hole 54, elongated opening 48, and pin hole 19 and thereby links knob 12 with inner sleeve 18, the elongated opening allowing the pin to slide along outer sleeve 14. In this manner, knob 12 is configured to slide inner sleeve 18 and auto-resetting component 27 along the longitudinal axis of outer sleeve 14 and thereby controls the engagement between indicator button 30 and the auto-resetting component.

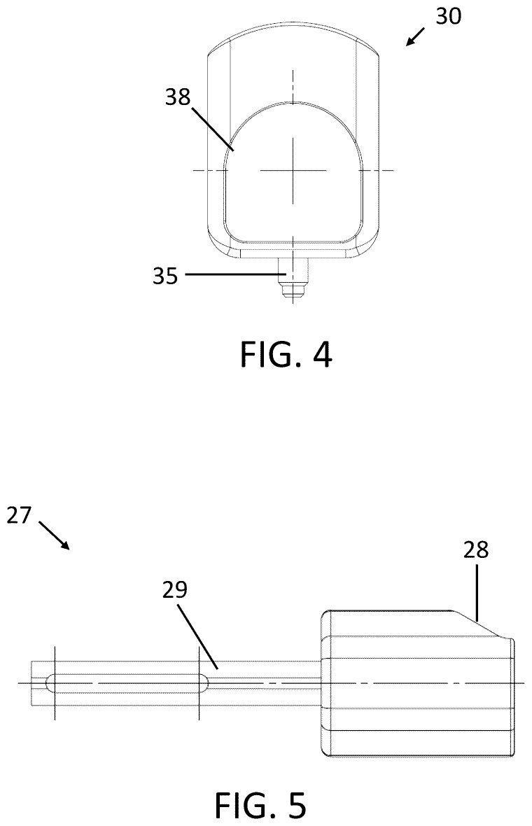

As shown in FIGS. 3-4, indicator button 30 includes an interior aperture 38 and an indicator spring 36. Indicator spring 36 is disposed in indicator compartment 45 of outer sleeve 14 and around a post 35 extending from indicator button 30. Indicator button 30 is also disposed within indicator compartment 45 and side opening 44 of outer sleeve 14 such that alignment tube 26 can extend through interior aperture 38 of the indicator button.

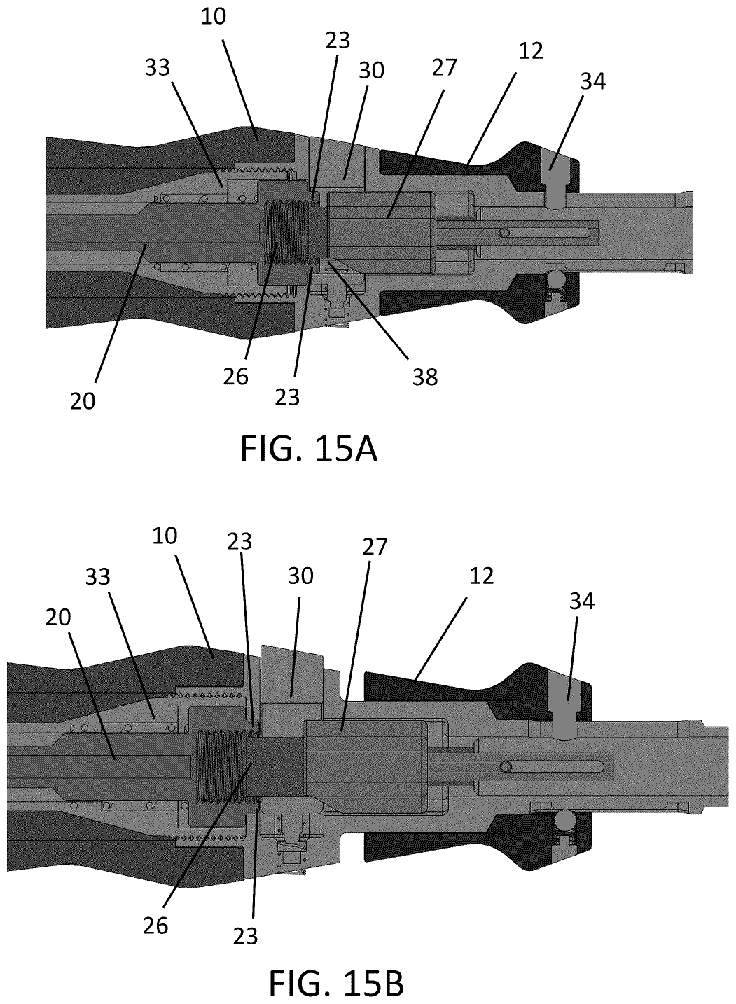

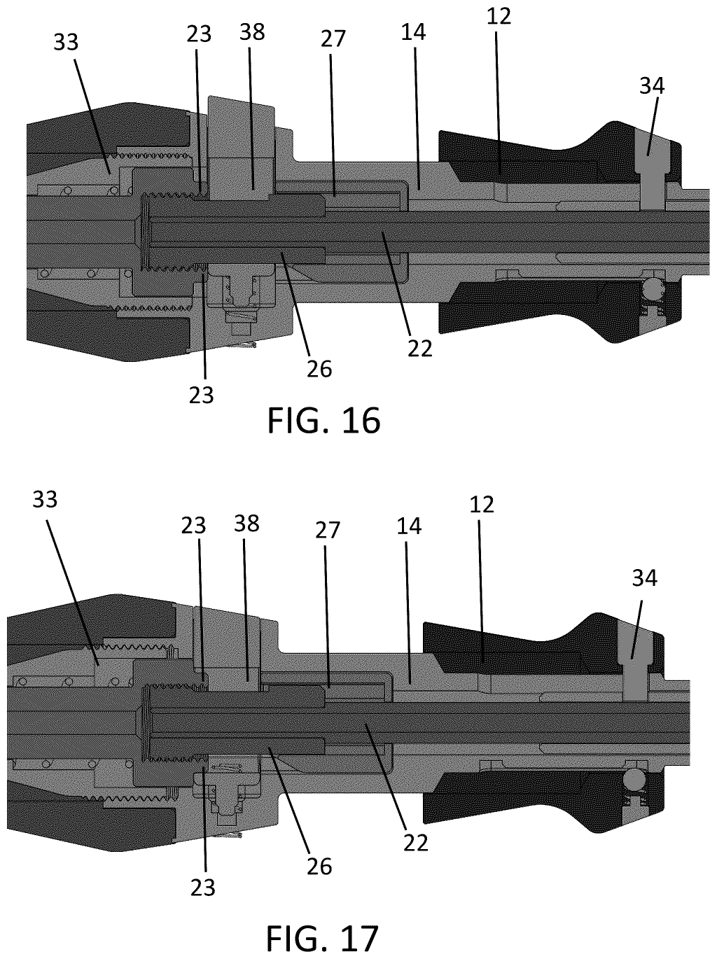

When indicator button 30 is in the non-indicating position (i.e., completely disposed within inserter 1) and knob 12 is fully retracted to abut flange 49, auto-resetting component 27 and lip portion 23 of setting shaft 20 are both partially disposed within interior aperture 38 of indicator button 30, as best shown in FIG. 15A. In other words, lip portion 23 is disposed within a proximal side of interior aperture 38 while auto-resetting component 27 is disposed within a distal side of the interior aperture. When indicator button 30 is in the indicating position and knob 12 is positioned at a distal end of elongated opening 48 (i.e., the locked position for the knob), neither lip portion 23 of setting shaft 20 nor auto-resetting component 27 are disposed within interior aperture 38 of indicator button 30, as shown in FIG. 16. In other words, when indicator button 30 is in the indicator position, nothing but alignment tube 26 of indicator rod 22 is located within interior aperture 38 which is what allows indicator button 30 to change positions. When indicator button 30 is in the non-indicating position and knob 12 is in the locked position, lip portion 23 is disposed within interior aperture 38 of the indicator button, but auto-resetting component 27 is not, as shown in FIG. 17. In other words, when inserter is in the locked, non-indicating position, only lip portion 23 of setting shaft 20 remains engaged with indicator button 30 to hold it in the non-indicating position.

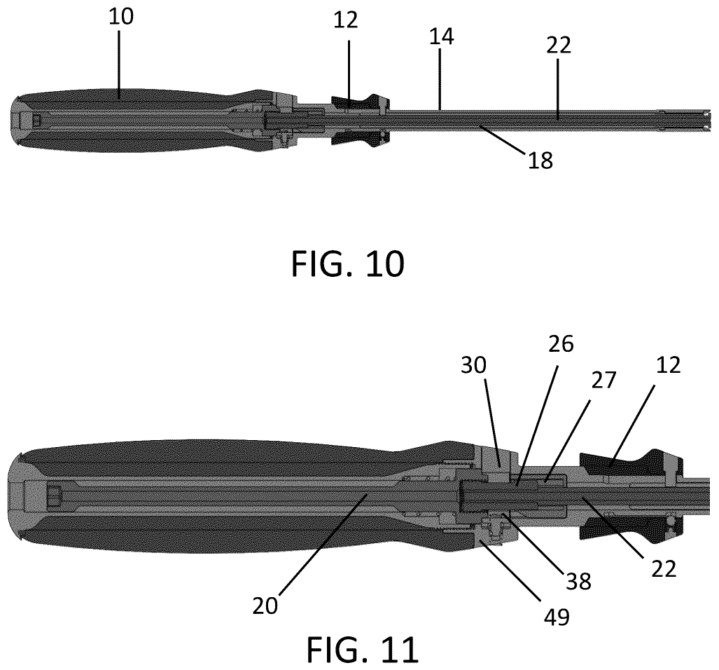

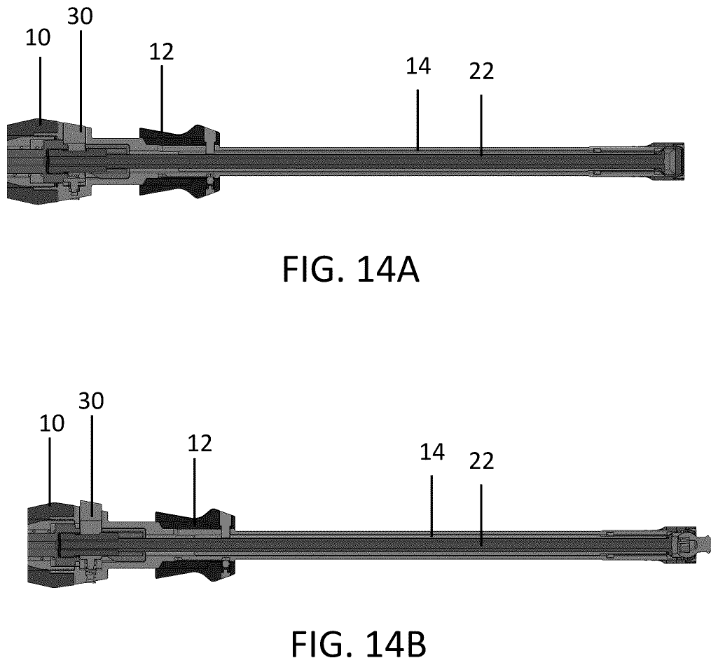

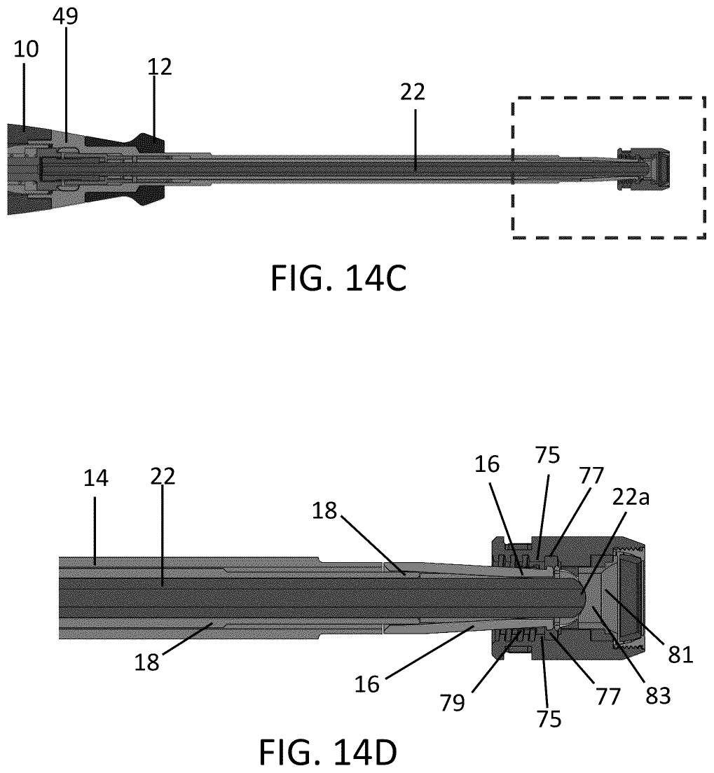

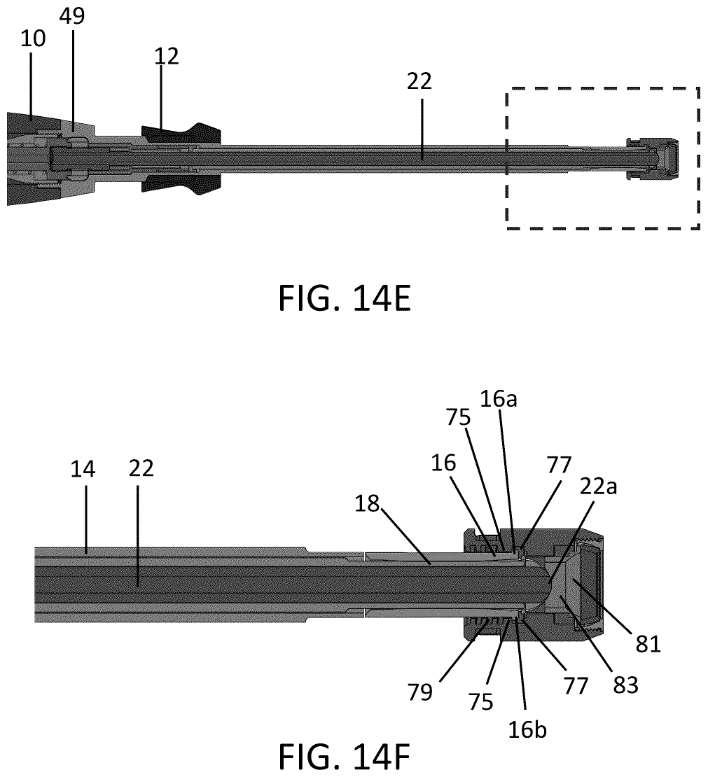

In another aspect, the present disclosure relates to a method of using an inserter instrument that has an indicator mechanism along with an auto-resetting component. With reference to FIGS. 10-17, inserter 1 may be placed in a locked, non-indicating position by sliding knob 12 toward the distal tip of outer sleeve 14. When transiting to the locked, non-indicating position, inserter 1 may be attached to a modular tulip head by splaying out attachment arms 16 into undercuts of the tulip head, as shown in FIGS. 14C-14F. Attachment arms 16 are forced to expand outward by inner sleeve 18 when knob 12 is slid distally which causes the inner sleeve to slide between the attachment arms and indicator shaft 22 and thereby expand or extend the attachment arms outwardly. Inserter 1 is detached from the tulip head by sliding knob 12 back to the unlocked position, as shown in FIGS. 14C-14D. In this manner, knob 12 can be utilized to attach and detach inserter 1 to a tulip head. In this same way, inserter 2, discussed below, is also configured to attach and detach to a tulip head.

In some instances, attachment arms 16 include attachment tips 16a and 16b that extend perpendicular to the length of the respective attachment arm from the distal end of thereof. In some such instances, when inserter 1 is attached to the tulip head, attachment arms 16 extend past threads 79 and shelf 75 of the tulip head such that insertion tips 16a and 16b splay into groove 77 of the tulip head when the attachment arms are in an expanded state, as shown in FIG. 14F. Threads 79 of the tulip head do not extend past shelf 75 and thus do not break or cut into shelf 75 and/or into groove 77. Groove 77 is not a threaded feature and revolves around the central axis of the tulip head. In other words, groove 77 is not a residual feature created from a manufacturing process used to cut threads 79 into the tulip head. The shape and placement of groove 77 and shelf 75 within the tulip head may be specifically designed to receive and engage with insertion tips 16a and 16b to allow for inserter 1 to better attach to or engage with the tulip head. In another embodiment, not shown, groove 77 may be any form of recess, hole, ledge, or shelf defined by the tulip head.

Once the tulip head is engaged to the distal end of inserter 1, the tulip head is attached to an implanted bone screw by providing a downward force to affix the two components together. While the present invention has applicability to many different types of modular pedicle screws, one such example is disclosed in U.S. patent application Ser. No. 18/011,612, the disclosure of which is hereby incorporated by reference herein. Upon proper placement and attachment of the tulip head, indicator button 30 is triggered by indicator shaft 22 pushing lip portion 23 of setting shaft 20 out of interior aperture 38 of the indicator button as distal end 22a of the indicator shaft contacts an implant 81 and is pushed proximally by the implant as the tulip head continues to be advanced over the implant. This proximal movement of indicator shaft 22 separates and disengages setting shaft 20 from indicator button 30. Once indicator button 30 is released from setting shaft 20, indicator spring 36 expands and thereby pushes the indicator button to the indicating position, as shown in FIG. 16, to indicate the tulip has been properly attached to the bone screw. In some instances, distal end 22a of indicator shaft 22 defines a rounded tip designed to be received by a saddle structure 83 defined by implant 81 such that the rounded tip conforms to the contours of the saddle structure.

At this point, the inserter is in the locked, indicating position. After proper attachment of the tulip head to the bone screw, the tulip is released from attachment arms 16 of inserter 1 by sliding knob 12 proximally toward handle 10 to retract inner sleeve 18 and the attachment arms from an expanded state. Knob 12 also causes auto-resetting component 27 to slide proximally such that the auto-resetting component engages and thereby repositions indicator button 30 as auto-resetting component 27 slides into interior aperture 38 of the indicator button, as shown in FIG. 15B. Once inserter 1 is in the unlocked position with indicator button 30 in the non-indicating position, lip portion 23 of setting shaft 20 is aligned with and reinserted into interior aperture 38 of the indicator button by setting spring 24 pushing the lip portion therein, as shown in FIG. 15B. With setting shaft 20 repositioned within the interior aperture, indicator button 30 will be held in the non-indicating position when knob 12 and auto-resetting component 27 slide forward (i.e., toward the distal end outer sleeve 14) into the locking position. In this manner, auto-resetting component 27 eliminates the hassle of manually resetting the indicator button and the potential confusion caused by a false indicator.

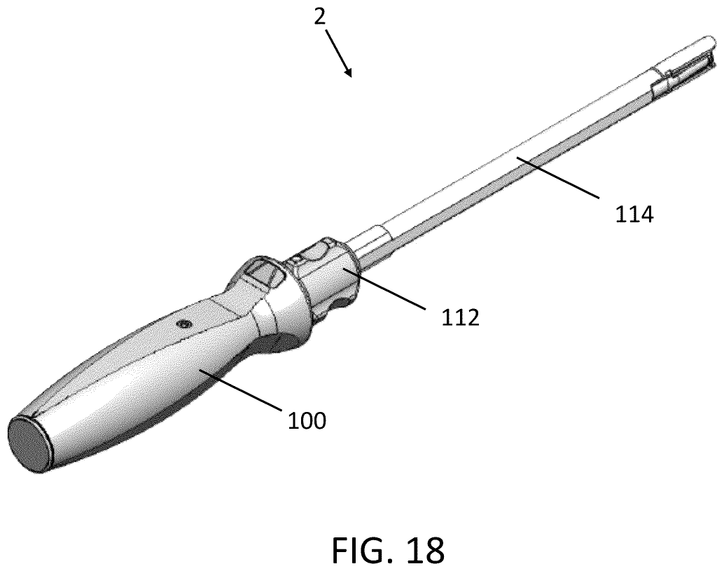

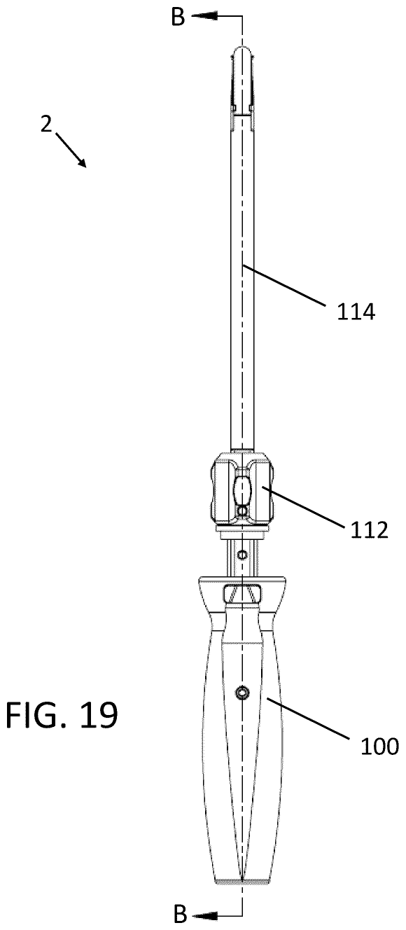

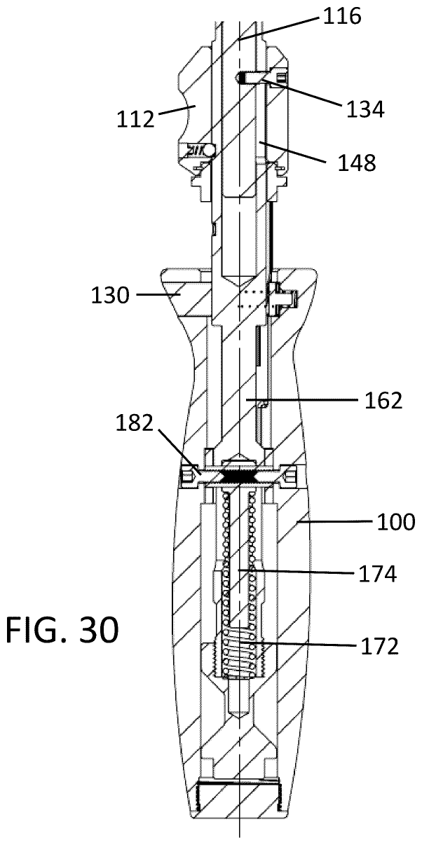

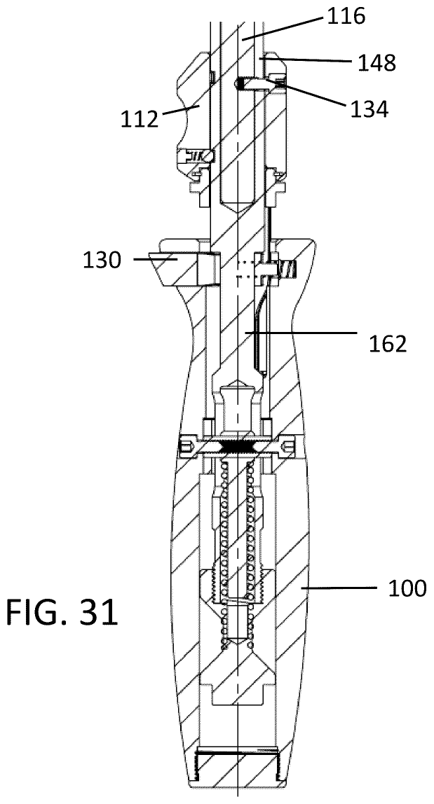

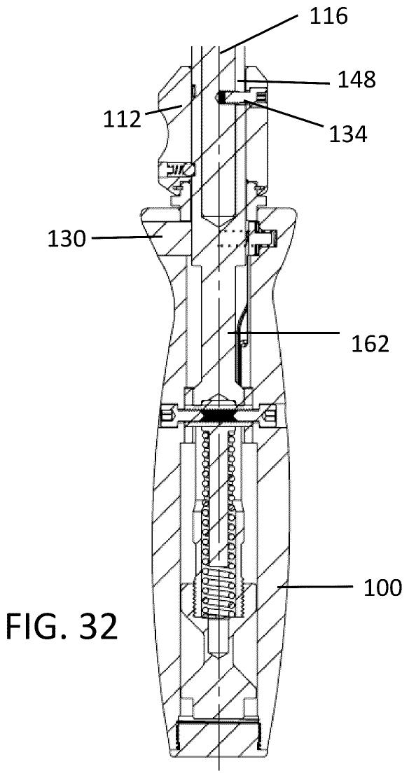

In another embodiment, as shown in FIGS. 18-31, an inserter 2 includes a handle 100 slidably connected to an outer sleeve 114, an inner shaft 118 slidably disposed within the outer sleeve, a knob 112 slidably disposed around the outer sleeve, and an auto-resetting component 127 that is also slidably disposed around the outer sleeve and attached to the knob. Inserter 2 also includes attachment arms 116 that are attached to a distal end of inner shaft 116. Attachment arms 116 are similar to attachment arms 16 in that they are designed engage and securely hold a modular tulip head when in an expanded state.

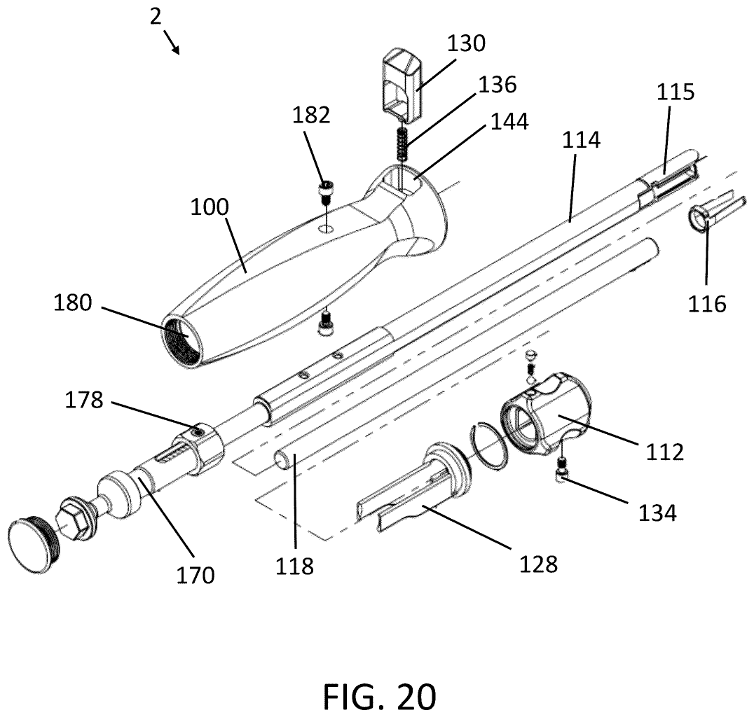

Handle 100 is a hollow tube with a proximal opening 100A, distal opening 100B, and a side opening 144 near the distal opening. As shown in FIG. 20, handle 100 includes an indicator button 130, handle screws 182, and a central cavity 180 that extends between distal opening 100B and proximal opening 100A of the handle. Indicator button 130 is disposed within an indicator slot 145 that extends into central cavity 180 from side opening 144 of the handle along with an indicator spring 136. Indicator button 130 defines an interior aperture 138 having a diameter large enough to receive outer sleeve 114 and auto-resetting component 127 at the same time. Handle screws 182 are positioned proximal to indicator button 130 to slidably connect handle 100 with outer sleeve 114, the handle screws are disposed partially in the handle and partially in the outer sleeve.

Outer sleeve 114 includes distal arms 115, a distal sleeve 160 that houses inner shaft 118, a proximal sleeve 164, and a narrow region 162 disposed between the distal and proximal sleeves. A distal opening 114A of outer sleeve 114 is defined by distal sleeve 160 such that distal arms 115 are disposed around the distal opening, and a proximal opening 114B of the outer sleeve is defined by proximal sleeve 164. Proximal sleeve 164 includes an actuating mechanism 170 and two oblong openings 166 that are aligned and disposed on opposite sides of the proximal sleeve such that an actuating pin 178 of the actuating mechanism 170 can extend therebetween.

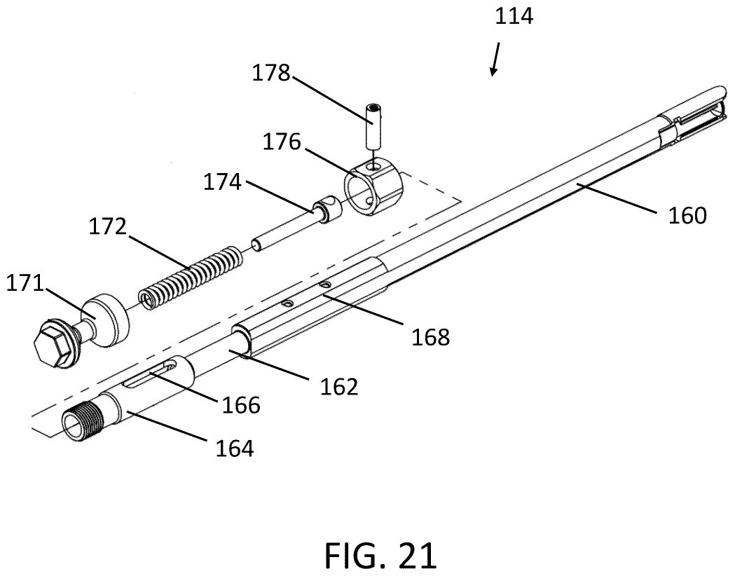

As shown in FIG. 21, actuating mechanism 170 of the outer sleeve further includes a threaded cap 171, an actuating spring 172 disposed around a spring rod 174, and an actuating collar 176 connected to the spring rod via actuating pin 178. Actuating spring 172 and actuating rod 174 are slidably disposed within proximal sleeve 164, and actuating collar 176 is slidably disposed around the proximal sleeve where the oblong openings 166 are located. Threaded cap 171 is threadably attached to the proximal end of proximal sleeve 164 such that the threaded cap holds actuating spring 172 within the proximal sleeve. Actuating spring 172 is a compression spring. When inserter 2 is assembled, actuating pin 178 is disposed within the pin holes in actuating collar 176 and spring rod 174 while extending between oblong openings 166 of proximal sleeve 164 to link the spring rod and actuating collar together. Additionally, each handle screw 182 is threadably attached to an end of actuating pin 178 such that handle 100 is linked to actuating collar 176 and spring rod 174. In other words, actuating mechanism 170 of proximal sleeve 164 are disposed within central cavity 180 of handle 100 such that handle screws 182 are aligned and attached to actuating pin 178. In this manner, actuating spring 172 is indirectly linked to handle 100 via handle screws 182 and actuating pin 178 such that the actuating spring resists proximal movement of handle 100 with respect to outer sleeve 114.

Knob 112 is a hollow annulus that is open at each end. The exterior of the knob 112 defines various ridge-like features with scalloped regions therebetween. Knob 112 includes pin screw 134 that extends perpendicular to the longitudinal axis of outer sleeve 114. Pin screw 134 extends from knob 112 into inner shaft 118 through an elongated opening 148 in distal sleeve 160 of outer sleeve 114 to connect the knob and inner sleeve together. In this manner, knob 112 and inner sleeve are linked together such that the knob can slide the inner sleeve within outer sleeve 114.

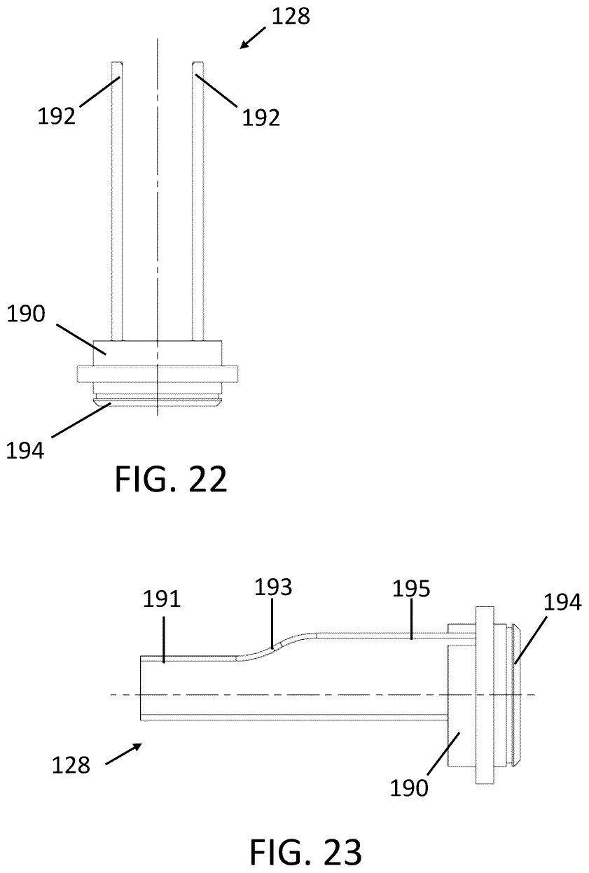

Auto-resetting component 127, as shown in FIGS. 22-23, includes two legs 192 extending from a ring 190 opposite to a threaded portion 194 of the ring, the two legs having a ramp-like profile with a short portion 191, a tall portion 195, and a sloped portion 193 disposed therebetween. These portions are sized such that they all can slide in and out of interior aperture 138 of indicator button 130. More specifically, the height of short portion 191 for each leg 192 corresponds to the diameter of narrow region 162 of outer shaft 114, and the height of tall portion of each leg 192 corresponds to the height of distal sleeve 160 immediately adjacent to the narrow region (i.e., expanded region 168). The distance between legs 192 corresponds to the outer dimensions of distal sleeve 160 of outer sleeve 114 such that the outer sleeve can fit therebetween. Threaded portion 194 of ring 190 is threadably attached to a proximal end of knob 112. In this manner, knob 112 and auto-resetting component 127 are linked together such that the position of the auto-resetting component is determined by the position of the knob.

In another aspect, the present disclosure relates to a kit that may include an inserter instrument having an auto-resetting component. Such kits may bone screw, modular tulip head, and an inserter instrument having an indicator button and an auto-resetting component, e.g., inserter 1, 2 described above. The kit may include multiple inserters of various sizes. There may be a variety of bone screws include in the kit having various designs and sizes. Additionally, such kits may include replacement parts for the inserter and tools to repair and maintenance.

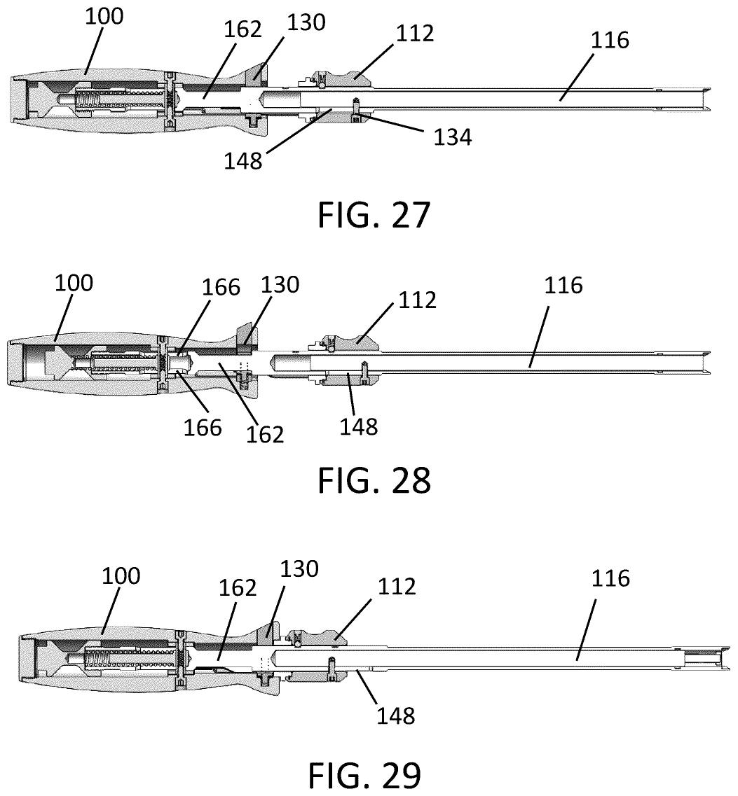

In another aspect, the present disclosure relates to another method of using an inserter instrument that has an indicator mechanism along with an auto-resetting component. With reference to FIG. 27, inserter 2 may be placed in a locked, non-indicating position by sliding knob 112 toward the distal tip of outer sleeve 114 to temporarily attach to a modular tulip head. When transiting to the locked, non-indicating position, inserter 2 may be attached to a modular tulip head by splaying out attachment arms 16 into undercuts of the tulip head similar to inserter 1, as shown in FIG. 14A. Once the tulip head is locked to the distal end of inserter 2, the tulip head is attached to an implanted bone screw by placing and attaching the tulip head onto the implanted bone screw, similar to what is shown in FIG. 14B. Once a proper attachment of the tulip head is thought to have been achieved, such attachment is checked by pulling handle 100 away from the bone screw against the resistance of actuating spring 172. If the attachment of the tulip head is incomplete, pulling handle 100 in this manner will result in separation of the tulip head and the implanted bone screw. If the attachment of the tulip head was properly achieved, pulling handle 100 will move actuating collar 176 and spring rod 174 proximally such that actuating spring 172 is compressed against cap 171. As actuating spring 172 is compressed, slide indicator button 130 slides with handle 100 proximally along outer sleeve 114 until the indicator button reaches narrow region 162 of the outer sleeve. Once narrow region 162 is the only portion of outer sleeve 114 positioned within interior aperture 138 of indicator button 130, indicator spring 136 pushes the indicator button through side opening 144 and into the indicating position, as shown in FIG. 28. At this stage, the attachment of the tulip head can be reconfirmed by pressing indicator button 130 back to the non-indicating position, sliding handle 100 back toward the tulip head, and repulling the handle until the indicator button actuates. In this manner, inserter 2 eliminates false indications of proper attachment by using an indicating mechanism that actuates only after the attachment of the tulip had as been affirmatively tested and providing the means to reconfirm that attachment after indicator has been triggered.

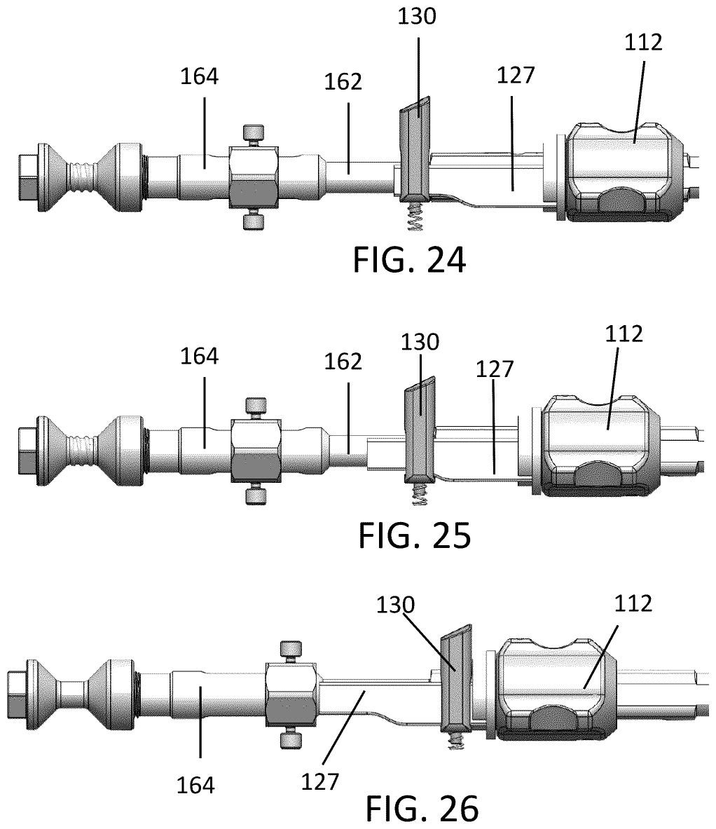

Once the attachment of the tulip head has been confirmed by the actuation of indicator button 130, inserter 2 is in a locked, indicating position. The tulip head is then released from attachment arms 116 by sliding knob 112 and auto-resetting component 127 proximally toward handle 100 to collapse the attachment arms from an expanded state. As auto-resetting component 127 slides proximally into interior aperture 138 of indicator button 130, sloped portion 193 of the auto-resetting component gradually pulls the indicator button into indicator slot 145 and back to the non-indicating position, as shown in FIGS. 24-26. In this manner, inserter 2 automatically resets indicator button 130 with auto-resetting component 127 as the tulip head is released with the retraction of knob 112.

It is contemplated that the components described herein for inserter 1 and inserter 2 can be made from a variety of materials. For example, the handles, shafts, sleeves, pins, screws, springs, and other various components of the inserters may be made from various plastics and/or metals such as stainless-steel or aluminum. Components like the handle, auto-resetting component, and the attachment arms may be made from both metal and plastic materials. Additionally, these components may be made from both additive and subtractive manufacturing techniques.

It is to be understood that the disclosure set forth herein includes any possible combinations of the particular features set forth above, whether specifically disclosed herein or not. For example, where a particular feature is disclosed in the context of a particular aspect, arrangement, configuration, or arrangement, that feature may also be used, to the extent possible, in combination with and/or in the context of other particular aspects, arrangements, configurations, and arrangements of the technology, and in the technology generally.

Furthermore, although the technology herein has been described with reference to particular features, it is to be understood that these features are merely illustrative of the principles and applications of the present technology. It is therefore to be understood that numerous modifications, including changes in the sizes of the various features described herein, may be made to the illustrative arrangements and that other arrangements may be devised without departing from the spirit and scope of the present technology. In this regard, the present technology encompasses numerous additional features in addition to those specific features set forth in the claims below. Moreover, the foregoing disclosure should be taken by way of illustration rather than by way of limitation as the present technology is defined by the claims set forth below.

1. A surgical instrument for inserting and attaching an implant, comprising:
an outer sleeve having a distal end and a proximal end, the distal end configured to receive a tulip head,
an indicator button adjacent the proximal end, the indicator button movable between a first position and a second position;
a shaft slidably disposed within the outer sleeve; and
an auto-resetting component slidably linked to the indicator button and the shaft, the auto-resetting component configured to automatically reposition the indicator button to the first position.

2. The surgical instrument of claim 1, wherein the shaft is configured to actuate the indicator button when the tulip head is securely attached to a bone screw component of the implant.

3. The surgical instrument of claim 1, wherein the auto-resetting component includes a reset ramp.

4. The surgical instrument of claim 3, wherein the reset ramp is slidably disposed within an aperture of the indicator button.

5. The surgical instrument of claim 1, wherein the auto-resetting component is disposed within the outer sleeve.

6. The surgical instrument of claim 1, wherein the auto-resetting component is disposed external to the outer sleeve of the surgical instrument.

7. The surgical instrument of claim 4, further comprising an inner sleeve disposed between the outer sleeve and the shaft such that the shaft is slidably disposed within the inner sleeve.

8. The surgical instrument of claim 1, wherein the auto-resetting component includes connector arms extending distally from the auto-resetting component such that the connector arms attach to a proximal side of an inner sleeve disposed between the outer sleeve and the shaft.

9. The surgical instrument of claim 4, wherein the indicator button includes an internal portion and an indicating portion, the internal portion defining the aperture of the indicator button.

10. The surgical instrument of claim 8, wherein the indicator button is attached to an indicator spring.

11. The surgical instrument of claim 10, wherein the indicator button and the indicator spring are positioned orthogonal to a longitudinal axis of the outer sleeve.

12. The surgical instrument of claim 7, wherein the inner sleeve includes at least two actuating arms extending from a distal end of the inner sleeve configured to engage a groove of the tulip head.

13. A method of inserting a modular tulip head using an inserter, comprising:
engaging the tulip head with the inserter;
placing the tulip head onto a bone screw;
triggering an indicator button to move to an indicating position after placement of the tulip head on the bone screw; and
retracting a knob of the inserter to an original position after the indicator button is triggered such that the indicator button is automatically reset to a non-indicating position by a reset component.

14. The method of claim 13, wherein the indicator button is automatically reset by sliding the reset component into an aperture of the indicator button.

15. The method of claim 13, wherein the engaging of the tulip head includes sliding the inner sleeve in a distal direction along the longitudinal axis of the outer sleeve and actuating arms extending from the inner sleeve to engage an inner groove of the tulip head.

16. The method of claim 15, wherein the triggering of the indicator button is done by displacing a setting shaft arranged to hold the indicator button in the non-indicating position while partially inserted into an aperture of the indicator button.

17. The method of claim 16, wherein the displacing of the setting shaft is caused by shifting an indicator shaft in the distal direction due to contact with an implant.

18. The method of claim 17, wherein the retracting of the inner sleeve causes the reset component to engage the aperture of the indicator button.

19. A surgical instrument for inserting and attaching a tulip head to a bone screw, comprising:
a sleeve having a distal end and a proximal end, the distal end configured to receive the tulip head;
an indicator button configured to actuate between an indicating position and a non-indicating position;
an inner shaft slidably disposed within the sleeve and configured to engage the tulip head;
a knob slidably disposed around the sleeve; and
a reset ramp linked to the knob, the reset ramp configured to automatically reposition the indicator button from the indicating position to the non-indicating position.

20. The surgical instrument of claim 19, wherein the reset ramp is disposed external to the sleeve.
